(19) United States
(12) Patent Application Publication
(10) Pub. No.: US 2021/0140980 A1
(43) Pub. Date: May 13, 2021

(54) SEPTAPEPTIDES ASSOCIATED WITH NEURODEGENERACY

(71) Applicant: SEPTA THERAPEUTICS, INC., Penticton (CA)

(72) Inventor: Diane VAN ALSTYNE, Penticton (CA)

(73) Assignee: SEPTA THERAPEUTICS, INC., Penticton, BC (CA)

(21) Appl. No.: 16/959,990

(22) PCT Filed: Jan. 3, 2019
(86) PCT No.: PCT/CA2019/050004

§ 371 Date: Jul. 2, 2020

Related U.S. Application Data
(60) Provisional application No. 62/613,621, filed on Jan. 4, 2018.

(51) Int. Cl. G01N 33/68; G01N 33/50; C07K 14/285; C07K 14/22; C07K 14/315; C07K 14/245; C07K 14/20; C07K 14/195; C07K 16/12

(52) U.S. Cl. CPC G01N 33/6896; A61K 38/00; C07K 14/285; C07K 14/22; C07K 14/3156; C07K 14/245; C07K 14/20; C07K 14/195; C07K 16/1207; C07K 16/1217; C07K 16/1275; C07K 16/1232; C07K 16/1296; G01N 2800/2821; G01N 2800/2835; G01N 2800/52; G01N 33/5058

(57) ABSTRACT

Methods and compositions useful in treating neurodegenerative disorders are based on septapeptides and extended forms thereof (or portions thereof) that exhibit chemokine activity with respect to microglial cell precursors.

### CROSS-REFERENCE TO RELATED APPLICATIONS

This application claims the benefit of U.S. Provisional Application No. 62/613,621, filed 4 Jan. 2018, which application is incorporated herein by reference.

### INCORPORATION BY REFERENCE OF SEQUENCE LISTING

The present application is being filed along with a Sequence Listing in electronic format. The Sequence Listing is provided as a file entitled 186182000140SeqList.txt, created Jan. 3, 2019, which is 6,835 bytes in size. The information in the electronic format of the Sequence Listing is incorporated by reference in its entirety.

### FIELD OF THE INVENTION

The invention is directed to compositions and methods related to the treatment of neurodegenerative disease. More specifically, it concerns use of septapeptide and septapeptide related moieties for identification of agents for treatment of such diseases as well as methods to treat and diagnose such conditions.

### BACKGROUND ART

AD is an age-related neurodegenerative disorder characterized by progressive loss of cognitive and executive functions. The main features of AD are the extracellular deposition of amyloid beta (Abeta) protein of unknown origin into the extracellular amyloid-plaques and the intracellular accumulation of tau protein in the form of neurofibrillary tangles. Hardy J., Selkoe D. J., Science (2002) 297, 353-356. The amyloid beta precursor protein (AbetaPP) is synthesized as a transmembrane glycoprotein, with the bulk of the molecule in the extracellular space. It is ubiquitous throughout the animal kingdom and its sequence is highly conserved. The AbetaPP may be cleaved by beta-secretase at the N-terminal end of the beta peptide sequence and goes on to produce the beta peptide itself through an additional proteolytic cleavage at the C terminal. Soluble beta-peptide is found normally, in non-AD patients, in the blood and cerebrospinal fluid. The reported sequences vary from 40, 42 or 48 amino acid residues. The commonly held view is that some part of the Abeta protein is the AD “trigger” and the neurons' tau protein tangles are the end result of the disease process. Hardy J., Neuron (2006) 52:3-13. Hickman, S., et al., Nature Neurosci (2018) 21:1-8, DOI:10.1038/s41593-018-0242-x.

Amyloid beta is known to induce microglial/astrocyte cell activation which in turn promotes inflammation through the release of proinflammatory mediators. Microglia/astrocytes are innate immune cells in the brain. They constantly scan the tissue, and respond to pathological signals to encapsulate pathogenic foci and remove apoptic cells/debris, much as the peripherally-derived macrophages do. However, activated microglia and peripherally-derived macrophages take on a proinflammatory role and generate proinflammatory mediators, such as TNF-alpha, Ill-beta, chemokines, complement factors and other neurotoxic factors. Hickman, S., et al., Nature Neurosci (2018) 21:1-8, DOI:10.1038/s41593-018-0242-x. Abeta has also been shown to activate hippocampal neurons from embryonic cells cultured from rat brains, through the activity expressed by the Abeta 1-40 sequence²⁶GSNKGAIIGLN³⁶ (SEQ ID NO: 4) at low concentrations of 0.1 nM.

Conversely, extraordinarily high concentrations over 10 OM were toxic and killed neurons, suggesting that Abeta may kill neurons if present in sufficiently high concentrations (Yankner, B A., et al., Science (1990) 250:279-282). Another Abeta domain, ¹³HHQK¹⁶ (SEQ ID NO: 5), has been shown to bind to microglia, thereby blocking plaque-induction of neurotoxicity by reducing inflammation (Glulian, D., J. Biol. Chem. (2016) 273, 29719-29726). Abeta has also been proposed as toxic to neurons through synaptic damage. Communication between nerve cells occurs at highly specialized cellular structures known as synapses. Loss of synaptic function is associated with cognitive decline in AD. There is still no clear mechanism to account for synaptic damage. The damage appears to be related to extrasynaptic glutamate released from an overabundance of astrocytes/microglia in response to Abeta¹⁻⁴² peptide. (Talantova, M, et al., P.N.A.S. (2013) USA June 17.doi: 10.1073/pnas.1306832110.)

It is not known whether the excess glutamate is derived from the over-stimulation of existing astrocytes/microglia in the presence of Abeta¹⁻⁴² or whether the Abeta contains chemokine activity which induces the formation of an overabundance of astrocytes/microglia from existing progenitor cells. However, there is agreement that deposition of intracellular plaques containing Abeta is a hallmark proteinopathy of the disease. Further, that a neuroinflammatory response involving polarized microglial activity, enhanced astrocyte reactivity and elevated pro-inflammatory cytokine and chemokine load has long been implicated in AD and proposed to facilitate the neurodegeneration characteristic of AD. (Minter, M R et al., J. Neurochem. (2016) 136(3):457-74.)

Most research in the past 25 years has focused on the amyloid hypothesis of AD pathogenesis and progression. However, recent studies of neurofibrillary tau pathology comprising tangles and threads, in a range of neurodegenerative disorders, have shown that the abnormal, intracellular, tau protein pathologies occurs through prion-like seed-dependent aggregation, in which intracellular amyloid-like fibril formation in neurons is observed. Hasegawa, M., Biomolecules (2016) 6(2):24-32. This school of thought suggests that intervention in the conversion of normal tau to abnormal forms and in cell-to-cell transmission of tau may be the key to development of disease-modifying therapies for AD and other dementia-related disorders. Wischik, C. M., et al., J. Alzheimers Dis. (2015) 44:705-720.

Prions have been defined as host-encoded cellular proteins with the ability to undergo structural isomerization to an infectious form, termed PrP Sc. The process is autocatalytic, with Prp Sc serving as a template for refolding of PrP C. Prusiner S B, (2007). Prions. In: Knipe, D M, et al., editors. Page 6/14 Fields Virology. 5th Ed. Philadelphia: Lippincott Williams & Walkins pp 3059-3092. Protein aggregation and amyloid formation are central pathologic events in a wide range of neurodegenerative illnesses, including Gerstmann-Straussler-Scheinker (GSS), AD, Parkinson's and prion diseases. One specific prion protein mutation, lacking a plasma membrane anchor, has been shown to cause GSS disease, characterized by widespread amyloid deposition in the brain and the presence of a short protease-resistant PrP fragment similar to those found in GSS patients. The PrP 23-88 peptide sequence was found to greatly increase disease progression when administered to mice. Stohr J., Proc. Natl Acad Sci USA (2011) 108(52):21223-21228. These findings strongly suggest that prion-mediated spread of neuronaldys function in the CNS represents a general biological phenomenon in amyloidosis that leads to neurodegeneration.

Another aspect of current thought relates to meningitis related homologous antigemic sequences (MRHAS) and MCP-derived muteins. A member of the MRHAS family can be defined as an amino acid sequence that is homologous to antigenic sites on the structural polypeptides (within the core and E2 membrane portion of Rubella Virus (RV), that are recognized by a monoclonal antibody (Mab) from the hybridoma RV-1. Members of the MRHAS family were also found to appear in one variant of the chemokine, human Monocyte Chemoattractant Protein 1 (hMCP-1). The MRHAS muteins range from 29% (2/7 matches, ex. Rubella proteins) to 57% (4/7 ex. L monocytogenes p60). Over such short stretches of sequence, these MRHAS's are of arguable significance in terms of true homology; computer generated align plots show no statistically significant relatedness. However, if one allows for charge conservative substitutions, some of the MRHAS muteins reach 71% similarity with the MCP protein septapeptide. Despite the relatively low level of sequence conservation, antibody cross-reactivity among infectious agents carrying MRHAS's suggests the possibility of a conserved structure of the septapeptide motif not necessarily reflected in the linear sequence (see FIG. 1). Also see U.S. Pat. Nos. 5,510,264 and 5,556,757.

Since the MRHAS's that appear on bacterial and viral organisms are homologous with the C-terminal septapeptide sequence found in monocyte attracting chemokine hMCP-1, it is apparent that these agents have incorporated these sequences into their proteins to attract monocytes to aid in CNS infection by trafficking these agents through the BBB to infect the CNS. The placement of the MRHAS in the surface exposed loops found in pathogenic gram negative bacteria outer membrane protein (OMP) surface exposed loops maximizes and facilitates the attraction of immature monocytes to the meningitis-causing bacteria. See FIG. 2.

The unexpected discovery of Mab cross-reactivity over various viral and bacterial species known to cause meningitis provides means for therapeutic and prophylactic treatments of meningitis. There is also significant homology of these antigenic sites with amino acid sequences in monocyte-attracting chemokines. Thus, as shown herein, these means as disclosed herein may be applied to diseases as diverse as meningitis, atherosclerosis, and AD wherein the pathogen or pathogenic mechanism includes one or more of these MRHAS's.

### The Significance of Long Term Restriction of Viral Replication in the CNS to Studies of Neurodegenerative Disorders, Such as AD.

### Activation of a Latent Viral Infection

The possibility of AD treatments' inadvertent activation of a latent, chronic viral infection has been confirmed when Abeta⁴² was used in an attempt to construct a vaccine to prevent AD. Subacute meningoencephalitis was observed in 6% of the patients treated with the vaccine, without relation to serum anti-Abeta⁴² antibody titers. This finding strongly suggests that some sequence in the Abeta protein may have entered the CNS and activated previously infected progenitor cells, thereby releasing mature virus particles which cause a detectable, viral-mediated meningitis. Orgogozo, J M, Neurology (2003) 61:46-54.

### AD-Related Sequelae as Long-Term Consequence of Chronic HIV Infection in the CNS

It has long been known that the CNS serves as a reservoir for the human immunodeficiency virus (HIV), even as Highly Active AntiRetroviral Therapy (HAART) has been maintained. However, as the HIV pandemic enters its third decade, the dementia associated with HIV has differed from the dementia of AD, and some patients on HAART with suppressed viral replication have started manifesting persistent cognitive deficits suggesting that HIV-associated dementia may be evolving. There are now deepening concerns that HIV patients will start presenting with AD, independent of any classical HIV encephalopathy that may be present. Lescure F X et al., Clin Infect Dis (2011) 52(2):235-243. Clifford D B, et al., Neurology (2009) 73:1982-1987. Further, the expression of MRHAS septapeptides in the HIV structure, such as QNQQEKN (SEQ ID NO: 6) (FIG. 1), in the viral envelope, further suggests that the presence of the virus itself may (1) augment the appearance of astrocytes/microglia in the CNS while it may (2) also activate the host Abeta response to the chronic HIV infection.

### AD-Related Sequelae as Long Term Consequence of Prion Infection in the CNS

Protein aggregation and amyloid formation are central pathologic events in a wide range of neurodegenerative illness, including AD, Parkinson's, and prion diseases like GSS, as discussed. The expression of MRHAS septapeptide QNNFVHD (SEQ ID NO: 7) in the prion protein (FIG. 1) which hastens the onset of neurologic illness accompanied by a profound CNS amyloidosis resembling GSS, when used to transmit the disease within peptide 60-70 (Prusiner, 2007, Prions, in: Knipe et al., editors. Fields Virology, 5^{th} Ed. Philadelphia). These findings implicate the prion protein in the generation of tangles inside neurons, initially proposed by Ghetti B, et al., Neurology (1989) 39(11):1453-61. This activity further suggests that the presence of the prion may (1) augment the appearance of microglia in the CNS while it may also (2) act on neurons leading to neurodegeneration.

All documents cited herein are incorporated herein by reference.

### DISCLOSURE OF THE INVENTION

The present invention is grounded in the understanding that septapeptides analogous to those associated infections that affect the brain, such as meningitis, are also relevant to conditions associated with neurodegeneration, such as Alzheimer's disease (AD). This is verified herein by the showing that such a septapeptide is associated with the amyloid beta plaques considered the hallmark of AD and that this septapeptide exhibits maturation effects on glial cells.

This invention provides novel septapeptides homologous with previously reported homologous antigenic amino acid sequences on regions of bacterial and viral agents known to cause meningitis and on chemokines known to attract monocytes. It was proposed that meningitis-causing organisms use their MRHAS to attract and infect a monocyte subset, which then transports the organism through the blood-brain-barrier (BBB) into the brain. The importance of the MRHAS in the pathogenicity of meningitis-causing E. coli K1 has since been confirmed by Mittal, et al., J Biol. Chem. (2011) 286(3):2183-2193. The E. coli K1 homologous sequence NNGPTHE (SEQ ID NO: 8) in the outer membrane protein (OMP) was changed to NN-AAA-THE (SEQ ID NO: 9) and NNGP-AAA (SEQ ID NO: 10), thereby preventing the bacteria from entering the CNS to cause meningitis.

It has now been found that the Abeta protein accumulates over time as an immune response to a chronic, viral infection in the CNS, employing stem-cell activation to amplify phagocytic astrocytes/microglia populations to eliminate the infectious agents and concomitant cell debris.

The invention provides a neurodegenerative paradigm whereby a peptide sequence found in Amyloid beta or in a viral agent established in a chronic CNS infection, contributes to the initiation of neurodegenerative diseases including AD. There is a significant and urgent need to develop a therapeutic capable of blocking such initiation. Another additional need is for diagnostic tests to indicate AD or other neurodegenerative diseases as well as for use in the development of vaccines to block the entry of viral agents into the CNS.

Thus in one aspect the invention is directed to a method for prophylactic or therapeutic treatment of a neurodegenerative condition in a subject which method comprises administering to said subject an agent that blocks the monocyte chemoattractant protein-1 (MCP-1) receptor on microglia precursor cells or that selectively binds to HHQKLVF (SEQ ID NO: 11).

In another aspect the invention is directed to a method to identify an agent for prophylactic or therapeutic treatment of a neurodegenerative condition in a subject which method comprises measuring the response of microglia precursor cells or stem cells to a septapeptide, an extended form thereof or binding portion thereof or extended form of said binding portion in the presence and absence of a candidate agent, wherein a candidate agent that diminishes the response of said cells to the septapeptide or portion or extended forms thereof is identified as an agent for prophylactic or therapeutic treatment of said neurodegenerative condition.

In another aspect the invention is directed to a method to identify an agent for prophylactic or therapeutic treatment of a neurodegenerative condition in a subject which method comprises contacting a candidate agent with a septapeptide or an extended form thereof and determining the presence or absence of any complex formed, whereby a candidate agent that forms said complex is identified as an agent for prophylactic or therapeutic treatment of said neurodegenerative condition.

In another aspect the invention is directed to a recombinant expression system for an extended septapeptide which comprises a nucleotide sequence encoding said extended septapeptide operably linked to heterologous control sequence to effect expression.

In another aspect the invention is directed to a vaccine for prevention of neurodegenerative conditions caused by infectious agents which comprises as active ingredient a septapeptide or an extended form thereof along with a suitable pharmaceutically acceptable carrier.

In another aspect the invention is directed to a method to determine the presence or absence of a neurodegenerative disease in a subject, which method comprises determining the presence or absence of a septapeptide or extended form thereof in the cerebral spinal fluid (CSF) of said subject, whereby the presence of said septapeptide or extended form thereof indicates the presence of neurodegenerative disease in said subject.

In another aspect the invention is directed to a binding moiety that is specifically reactive with a septapeptide or extended form thereof wherein the septapeptide is selected from the group consisting of those shown in Table 4.

### BRIEF DESCRIPTION OF THE DRAWINGS

FIG. 1 shows hMCP-derived septapeptides in some meningitis-causing bacteria and viruses, prions and amyloid beta, showing sequences relevant to the initiation of AD. These sequences may provide useful targets for the preparation of pharmaceutical formulations designed to block MCP activity.

FIG. 2 shows pathogenic gram negative bacteria outer membrane protein (OMP) surface exposed loops, with the location of septapeptide Meningitis-Related Homologous Antigenic Sequences (MRHAS) shown in red. This external placement of MRHAS ensures maximal chance for contact with, and infection of, the appropriate subset of stem cells destined for entry into the CNS.

FIG. 3 shows positive Ca⁺² mobilization response to MCP-1 in THP-1 cells, illustrating the nature of the positive increase in the relative fluorescence intensity (RFI) over time, in response to exposure to MCP-1.

FIG. 4 shows positive Ca⁺² mobilization response to QQTAPKA (SEQ ID NO: 12) (L. monocytogenes) in THP-1 cells. The Listeria-derived MRHAS uses a different receptor than the native MCP-1, shown by the lack of subsequent desensitization to MCP 1.

FIG. 5 shows positive Ca⁺² mobilization response to QVQNNKP (SEQ ID NO: 13) (H. Influenzae type b) in THP-1 cells. The H. influenzae-derived MRHAS elicits a low, positive RFI of 2.0 but blocks activation by the Listeria-derived MRHAS, indicating that these two peptides share a common receptor.

FIG. 6 shows negative Ca⁺² mobilization response to QQQPPKA (SEQ ID NO: 14) (S. pneumoniae) in THP-1 cells. The streptococcal-derived MRHAS elicits no mobilization response at all, yet still blocks activation by the Listeria-derived MRHAS, indicating that these two peptides share a common receptor. This difference in binding activity to different receptors may guide the strategy in developing a pharmaceutical formulation for the treatment of AD.

FIG. 7 shows rat glial (RG) stem cells grown in monolayers in continuous culture, showing phase contrast, unstained (A.) Untreated monolayer of RG stem cells and B. RG stem cell monolayer treated with 100 uM diBcAMP. Greater than 99% of cells transformed into mature glia. This transient effect was greatest at 24 hours.

FIG. 8 shows RG stem cell monolayer treated with 100 uM diBcAMP shown A. as brightfield; B. stained with anti-DAPI antibody to identify nuclei; C. stained with anti-CD11b antibody to identify microglia (staining green); D. stained with anti-GFAP antibody to identify astrocytes (staining red).

FIG. 9 shows RG stem cell monolayer treated for 72 hours with Abeta peptide HHQKLVFFAE (SEQ ID NO: 15) at 10⁻⁷M. This transient effect was greatest at 72 hours. A. stained with anti-DAPI antibody to identify nuclei; B. as unstained cells, under phase contrast, showing 27% maturation of the stem cells in the monolayer. A pharmaceutical formulation which could block the Abeta-derived activity would be a candidate for the development of a therapeutic to treat AD.

FIG. 10 shows a schematic representation of the invention paradigm for neurodegenerative pathology.

### MODES OF CARRYING OUT THE INVENTION

The invention relates to a new understanding of the relationship between infection and neurodegeneracy as well as neurodegenerate conditions resulting from other causes. This paradigm is summarized in FIG. 10. As shown, an infection of the central nervous system effects inflammation which leads to a microglial population explosion resulting in a cell mediated immune response (CMI). The infection results from the chemokine activity of viral and prion infectious agents shown in the diagram as HMCP-1. This also results, in somewhat a roundabout way, in amyloid beta accumulation which further generates chemokine activity resulting in a microglial population expansion. The glia initiate phagocytic activity clearing Abeta deposits, infectious organisms and cell debris, but after prolonged exposure they also release a variety of cytotoxic mediators such as TNF-alpha, IL-1-beta chemokines, complement factors and other neurotoxic factors that are lethal to surrounding cells. This results in development of neurodegenerative diseases.

### Definitions

A “septapeptide”—a 7-amino acid peptide or peptidomimetic thereof that has sufficient homology to the Abeta peptide HHQKLVF (SEQ ID NO: 11) to elicit antibodies cross reactive with said Abeta peptide when administered to a mammalian subject.

An “extended form of a septapeptide” is a 7-amino acid peptide extended by additional amino acid sequence wherein said 7-amino acid peptide has sufficient homology to the Abeta peptide HHQKLVF (SEQ ID NO: 11) extended by the same additional amino acid sequence to elicit antibodies cross reactive with said extended Abeta peptide when administered to a mammalian subject. Typically the additional amino acid sequence is of 2-20 or 5-20 amino acids at each of one or both of the N- and C-termini.

Of course the septapeptides and extended forms thereof include HHQKLVF (SEQ ID NO: 11) itself or its extended forms.

The designations “a” or “an” indicate one or more than one unless otherwise clear from context.

The invention includes binding moieties specifically reactive with certain septapeptides or their extended forms. These moieties include aptamers and antibodies.

As used herein, the term “antibody” or “mAb” includes immunoreactive fragments of traditional antibodies or mAbs that still retain immunospecificity or antigen-binding such as Fab, F(ab′)₂, Fᵥ fragments, and single-chain antibodies in which the variable regions of heavy and light chain are directly bound without some or all of the constant regions. Also included are bispecific antibodies which contain a heavy and light chain pair derived from one antibody source and a heavy and light chain pair derived from a different antibody source. Similarly, since light chains are often interchangeable without destroying specificity, antibodies composed of a heavy chain variable region that determines the specificity of the antibody combined with a heterologous light chain variable region are included within the scope of the invention. Chimeric antibodies with constant and variable regions derived, for example, from different species and species-ized forms are also included.

The critical amino acid sequences of the variable regions that determine specificity are the CDR sequences arranged on a framework which framework can vary without necessarily affecting specificity or decreasing affinity to an unacceptable level. Definition of these CDR regions is accomplished by art-known methods. Specifically, the most commonly used method for identifying the relevant CDR regions is that of Kabat as disclosed in Wu, T. T., et al., J. Exp. Med. (1970) 132:211 250 and Kabat, E. A., et al. (1983) Sequence of Proteins of Immunological Interest, Bethesda National Institute of Health, 323 pages. Another similar and commonly employed method is that of Chothia, published in Chothia, C., et al., J. Mol. Biol. (1987) 196:901 917 and in Chothia, C., et al., Nature (1989) 342:877 883. An additional modification has been suggested by Abhinandan, K. R., et al., Mol. Immunol. (2008) 45:3832 3839. The present invention includes the CDR regions as defined by any of these systems or other recognized systems known in the art.

The specificities of the binding of the mAbs of the invention are defined, as noted, by the CDR regions mostly those of the heavy chain, but complemented by those of the light chain as well (the light chains being somewhat interchangeable). Therefore, the mAbs of the invention may contain the three CDR regions of a heavy chain and optionally the three CDR's of a light chain that matches it. Because binding affinity is also determined by the manner in which the CDR's are arranged on a framework, the mAbs of the invention may contain complete variable regions of the heavy chain containing the three relevant CDR's as well as, optionally, the complete light chain variable region comprising the three CDR's associated with the light chain complementing the heavy chain in question. This is true with respect to the mAbs that are immunospecific for a single epitope as well as for bispecific antibodies or binding moieties that are able to bind two separate epitopes.

Bispecific binding moieties may be formed by covalently linking two different binding moieties with different specificities. Multiple technologies now exist for making a single antibody-like molecule that incorporates antigen specificity domains from two separate antibodies (bi-specific antibody). Suitable technologies have been described by MacroGenics (Rockville, Md.), Micromet (Bethesda, Md.) and Merrimac (Cambridge, Mass.). (See, e.g., Orcutt, K. D., et al., Protein Eng. Des. Sed. (2010) 23:221 228; Fitzgerald, J., et al., MAbs. (2011) 1:3; Baeuerle, P. A., et al., Cancer Res. (2009) 69:4941 4944.) For example, the CDR regions of the heavy and optionally light chain derived from one monospecific mAb may be coupled through any suitable linking means to peptides comprising the CDR regions of the heavy chain sequence and optionally light chain of a second mAb. If the linkage is through an amino acid sequence, the bispecific binding moieties can be produced recombinantly and the nucleic acid encoding the entire bispecific entity expressed recombinantly. As was the case for the binding moieties with a single specificity, the invention also includes the possibility of binding moieties that bind to one or both of the same epitopes as the bispecific antibody or binding entity/binding moiety that actually contains the CDR regions. The invention further includes bispecific constructs which comprise the complete heavy and light chain sequences or the complete heavy chain sequence and at least the CDR's of the light chains or the CDR's of the heavy chains and the complete sequence of the light chains.

The mAbs useful in the invention may be adapted to species to be treated—e.g. humanized.

### Applications

The invention includes vaccines and pharmaceutical compositions and methods of administering them.

The invention includes active agents useful in therapy and prophylaxis for any subject that is susceptible to neurodegenerative conditions. Typically, the subject is human.

The active agents of the invention may be administered in a variety of ways, as pharmaceutical compositions with typical excipients. Compositions that comprise micelles or other nanoparticles of various types may be useful for extending the duration of treatment. Any peptide-based agent may be administered as the encoding nucleic acids either as naked RNA or DNA or as vector or as expression constructs. The vectors may be non-replicating viral vectors such as adeno associated virus vectors (AAV) or the encoding nucleic acid sequence may be chemically or physically delivered into cells (Suskovitch, T. J., and Alter, G., Expert Rev Vaccines (2015) 14:205-219).

The agents are administered in a variety of protocols, including intravenous, subcutaneous, intramuscular, topical with suitable penetrants, inhaled and oral or by suppository.

### Production of Active Agents

The septapeptides of the invention may be produced directly using known peptide synthesis techniques. It may be preferable especially for the extended forms to produce these recombinantly using known techniques. Thus, with regard to the extended forms described herein, the invention includes nucleic acid molecules comprising nucleotide sequence encoding them, as well as vectors or expression systems that comprise these nucleotide sequences, cells containing expression systems or vectors for expression of these nucleotide sequences and methods to produce these peptides by culturing these cells and recovering the peptides produced. Any type of cell typically used in recombinant methods can be employed including prokaryotes, yeast, mammalian cells, insect cells and plant cells. Also included are human cells (e.g., muscle cells or lymphocytes) transformed with a recombinant molecule that encodes the extended forms of the septapeptides.

Typically, expression systems for the peptides of the invention include a nucleic acid encoding said protein coupled to control sequences for expression. In many embodiments, the control sequences are heterologous to the nucleic acid encoding the peptides.

### Determination of Chemokine Activity

In order to identify agents useful in therapy or prophylaxis of neurodegenerative conditions, competitive assays involving assessment of chemokine activity of candidate agents with septapeptides are employed.

Chemokine activity can be detected in two ways. Chemokine MCP-1 may express mitogen-like effects on monocytes in culture. Addition of the intact chemokine or the active site peptide can cause transient, differentiation-like events terminating after 72 hours in culture. Activity is scored upon visual examination of cells in culture (The Cytokine Handbook, 2nd ed., A. Thompson, Editor. Academic Press, 1994).

Alternately, chemokine activity can be measured more quantitatively using Ca⁺² mobilization. Chemokines including MCP-1, -2 and -3 bind to several transmembrane, G-protein-linked receptors on the surface of monocytes, macrophages and microglia. Ligand binding induces signal transduction through the receptor-associated heterotrimeric G-proteins, leading to the generation of free Ca⁺² in the cytoplasm of chemokine-stimulated cells. The Ca⁺² rise is transient, and can be measured using a spectrofluorimeter. (The Cytokine Handbook) A typical readout is represented in FIG. 3, showing a positive Ca⁺² mobilization response to MCP-1 in THP-1 cells. THP-1 monocytic cells were loaded with the Ca⁺² indicator dye Indo-1AM and their cytoplasmic Ca⁺² content was measured during laser excitation. As Ca⁺² increases in the cytoplasm, the relative fluorescence intensity (RFI) of the dye increases in real time, exciting the detectors in the spectrofluorimeter. For example, after MCP-1 challenge of THP-1 cells, the RFI increases rapidly, and returns to baseline after about 60 seconds, as exemplified by the “spike” in the trace in FIG. 3 (the smaller peak before the large spike is an artifact generated by the introduction of the sample). Because the heterotrimeric G-proteins involved in the generation of the Ca⁺² signal are both rapidly dissociated and deactivated by phosphorylation, the increase in cytoplasmic Ca⁺² after ligand engagement can be desensitized. Therefore, challenging the cells a second time with MCP-1 would not result in a second spike, nor would a Ca⁺² spike result from subsequent challenge by another ligand which engages the same MCP-1 receptor

Progenitor glial cells, preferably in monolayer form in tissue culture, are required in order to detect and measure chemokine activity of specific polypeptides. Such indicator cells have been established employing intracerebral injections of kainic acid to cause a profound loss of neurons at the injection site. in adult rat brain. Olney, J. W, et al., Brain Research (1974) 77:507-512. Coyle, J. T. and Schwarcz, R., Nature (Lond.) (1976) 263:517-519. Evidence has been presented for the occurrence of an active gliosis at the injection site. Singh, V. K. et al., Brain Research (1978) 146:195-199. Gliosis was confirmed when glial cells from kainic acid-lesioned brains of adult rats could be maintained in continuous cell cultures. The Rat Glial (RG) cultures were shown to exhibit the astrocyte-specific, cytolasmic glial fibrillary acidic protein (GFAP), and to be negative for 3 neuronal enzymic markers: choline acetyltransferase, tyrosine hydroxylase and glutamic acid decarboxylase. Singh, V. K. and Van Alstyne, D., Brain Research (1978) 155:418-421.

The RG cells were further characterized using dibutyryl cyclicAMP (dBcAMP), shown to promote a maturation-stimulating effect on embryonic progenitor cells in vitro. Shipiro, D. L., Nature (Lond.) (1973) 241: 203-204. The RG cell derived from the striata of injured adult rat brain exhibit fetal, progenitor characteristics. Prior to treatment, the RG monolayers were composed of large, flat cells with ill-defined junctions and lacked cellular processes, characteristic of stem cells in general. In the presence of 1 mM dB-cAMP, virtually all RG cells underwent morphological transformation becoming smaller, and denser, in addition to forming several processes per cell. Two cell-type-specific markers could be detected in dB-cAMP-treated cultures: the surface oligodendrocyte-specific galactocerebroside (GC) moiety was demonstrated to be present on less than 10% of the cells, whereas the intracellular astrocyte-specific GFA protein could be detected in 80% of the population. Van Alstyne, D., et al., Neuroscience Letters (1983) 40: 327-332.

The RG cell line described here has also been employed as a useful susceptible host cell line for the cultivation of those viruses which may require primary fetal glial cells for growth, the isolation and propagation of slow viruses associated with a variety of neurological, degenerative brain disorders and for the study of viral replication in the CNS. When RG cells were infected with RV, only 5 of the 7 viral proteins could be detected intracellularly, indicating restricted viral replication in these progenitor cells. The same 5 viral proteins continue to be detected upon continued subculture of the infected RG cells, while no mature virus could be detected in culture media at any tune. Pope, D. D. and Van Alstyne, D., Virology (1981) 113:776-780. Upon exposure to dBcAMP, infected RG cells, underwent differentiation, and a concomitant activation of normal viral replication. Viral peptides during restricted replication clustered around the nuclear membrane, at the point of the construction of the cytoskeleton required for differentiation and for viral transport to the outer membrane intracellular viral proteins over long periods on time in vivo, thereby establishing a chronic infection in the CNS. Van Alstyne, D. and Paty, D. W., Virology (1983) 124:173-180.

Agents that Block the MCP-1 Receptor

The invention includes methods to treat neurodegenerative diseases that employ agents that block the MCP-1 receptor on microglia precursor cells or on stem cells. Among these agents are shorter forms of the septapeptides represent positions 1-3 or 1-4 which represent the binding portions but not the activating portions of these peptides, as shown in FIG. 1. These include peptidomimetics of these shorter peptides. In particular, such tri- and tetrapeptides include HHQ and HHQK of the Abeta septapeptide or peptidomimetics thereof. (Such agents also include the chaperone domain of BRIOCHS protein and resveratrol or other related flavonoid. Such related flavonoids are described in Zeinali, M., et al., DOI:10.1016/biopha.2017.06.003 (2017)). FIG. 1 summarizes the hMCP-1-derived septapeptides in some meningitis-causing bacteria and viruses, and in prions and amyloid beta. FIG. 1 also proposes 2 sites for stem cell activation by hMCP-1-derived muteins: a tripeptide binding site and a tripeptide activation site whose relative positions are governed by proline residues which form bends in linear polypeptides. FIG. 2 illustrates the location of the MRHAS septapeptides on the surface-exposed loops of outer membrane proteins (OMPs) of meningitis-causing gram negative bacteria. Their external positioning permits maximum exposure to appropriate receptors on stem cells.

Additional homologs from other infectious agents (viruses, bacteria, etc.) or their components may be identified based upon their sequence homology with members of the MRHAS family. In practice, such homologs may be identified by reference to the MRHAS occurring in human Monocyte Chemoattractant Protein-1 (hMCP-1, QTQTPKT (SEQ ID NO: 23)). This method can be applied to other infectious agents or their components that are yet to be discovered. For example, a new virus, bacterium or related protein component that enters or resides in the CNS may align their amino acid sequences with those of the MRHAS in hMCP1, to obtain maximum homology. Such an alignment has been discovered in the amyloid beta protein known to accumulate in the brains of AD patients. The concomitant expression of increasing levels of chemokine activity in the brains of AD patients may be linked to the initiation of cell death associated with AD.

These comparisons suggest that the homologous septapeptide (MRHAS) sequences common to meningitis-causing bacteria and viruses, to prions, and to amyloid beta proteins may all express the same chemokine activity. In the CNS, the amyloid beta septapeptide may act to activate glial progenitor cells to form phagocytosing astrocytes/microglia in response to chronic viral infection. In addition, any established chronic viral infection would also likely express its own chemokine activity in order to facilitate its continued infection (Van Alstyne and Paty, Virology (1993) 124:173-180).

The HHQKLVF (SEQ ID NO: 11) sequence of Abeta would likely be present in vast excess, thereby rendering it the best target for a formulation to block activating activity. However, complete protection against the development of AD may also require the blocking of any MRHAS activity contributed by chronic viral infection, as well.

### EXAMPLES

The following examples illustrate but do not limit the scope of the invention.

### Example 1

### MCP-Signaling in THP Monocytic Stem Cells by Some Bacterial MRHAS Septapeptides

Some of the bacterial septapeptide MRHAS's (Sigma) were tested for their ability to increase cytoplasmic Ca⁺², and to desensitize subsequent Ca⁺² mobilization by MCP-1 and heterologous septapeptides. Some of the peptides acted as mild Ca⁺² agonists on their own. These included QQTAPKA (SEQ ID NO: 12) of L. monocytogenes (Figure. 4) and QVQNNKP (SEQ ID NO: 13) from H. influenzae type b (FIG. 5). While the positive control hMCP-1 and the L. monocytogenes-derived septapeptide QQTAPKA (SEQ ID NO: 12) both showed positive Ca⁺² responses, they clearly use different receptors as shown by the lack of subsequent desensitization to MCP-1 shown in FIG. 4.

FIG. 5 shows a weakly positive mobilization response to the H. influenzae type b-derived peptide QVQNNKP (SEQ ID NO: 13). However, even a weak response was sufficient to block the binding of the L. monocytogenes-derived peptide QQTAPKA (SEQ ID NO: 12), indicating a shared receptor.

The QQQPPKA (SEQ ID NO: 14) sequence from S. pneumoniae did not induce a Ca⁺² signal on its own, but may possess some ability to block Ca⁺² mobilization in response to QQTAPKA (SEQ ID NO: 12) from L. monocytogenes (FIG. 6). All randomized negative control septapeptide sequences were negative. None of the septapeptides appeared to enhance the intrinsic Ca⁺² mobilizing activity of native MCP-1.

TABLE 1
Signaling activity of some bacterial septapeptide
MRHAS's in peripheral monocytes

| ORGANISM | MRHAS | Ca⁺² flux | DESENSITIZATION |
|---|---|---|---|
| L. monocytogenes | QQTAPKA | positive | negative for MCP-1 |
| | (SEQ ID NO: 12) | | |
| H. influenzae type b | QVQNNKP | positive | positive for QQTAPKA |
| | (SEQ ID NO: 13) | | (SEQ ID NO: 12) |
| S. pneumoniae | QQQPPKA | negative | positive for QQTAPKA |
| | (SEQ ID NO: 14) | | (SEQ ID NO: 12) |

All randomized septapeptide sequences were negative.

In summary, certain of the MRHAS free septapeptide muteins may act on their own as mild agonists of Ca⁺² mobilization in THP-1 cells, progenitor cells of circulating peripheral macrophages. Their signal transduction activity may be mediated through a single (or a single class) of receptor, but the data do not link this with the MCP-1 receptor on THP-1 cells. These data support the proposal that infectious organisms may conserve and employ these sequences in order to facilitate their transport through the BBB to infect the CNS. These data further support the hypothesis that the septapeptides represent muteins of the MCP active site for monocyte stem cell activation.

### Example 2

### MCP-Signaling in Rat Brain Glial (RG) Cells by Some Abeta-Derived MRHAS Septapeptides

1. Rat Glial (RG) Progenitor Cells Established in Continuous Culture.

Since no appropriate CNS-derived cells are available for the detection of Ca⁺² mobilization, a continuous cell line of glial stem cells was established in culture in order to detect mitogenic-like effects scored upon visual examination. Glial cells from normal brain following kainic acid-induced lesions were established in continuous cell culture as previously described by Singh V K and Van Alstyne D., Brain Res (1978) 155:418-421.) Briefly, 1 ul of a solution of kainic acid monohydrate (Sigma), containing 5 nmole kainic acid in 0.9% saline, was injected into rat striatum under anaesthesia. Rats were anaesthetized and decapitated 5 days following injection and the brains were harvested immediately and placed in a sterile glass culture dish. The striatum from the injection site was dissected, placed in MEM (Gibco) containing 0.2% glucose, 10% fetal calf serum, 5 units/ml penicillin, 5 ug/ml streptomycin and then further minced using a sterile scalpel. The culture dish was then incubated at 37° C. in a humidified atmosphere containing 5% CO₂. Each day for the first 3-4 days, the unattached tissue fragments were removed, washed once in fresh medium and returned to the culture dish until a complete monolayer was formed. The resulting monolayers were then subcultured every 5-6 days after trypsinization. The monolayers were treated with 0.25% trypsin (Gibco) for 5 minutes at 22° C. The cells were collected by low speed centrifugation, washed twice with complete growth medium, counted and replated in glass culture dishes. Once the cell line was established in glass dishes, the cells were then adapted and transferred to plastic culture dishes.

The mitogenic effect of diBcAMP on RG monolayers is shown in FIGS. 7A (untreated) and 7B (treated) cells after 24 hours. The transient effect shows that greater than 99% of the RG cells in the monolayer converted to mature cells, indicating that these cells will be appropriate for use as indicator cells to test MRHAS peptides. Mature cells were characterized using ThermoFisher Scientific CD11b and GFAP (Glial Fibrillary Acidic Protein) monoclonal antibodies AlexaFluor commonly used to detect microglial and astrocyte-specific markers respectively. (Tran, E., et al. J. Neuroimmunology 74 (1997) 121-129). FIG. 8 shows identical cells under brightfield illumination, with positive staining for microglial and astrocyte markers (CD11b and GFAP markers respectively. The identification of the majority of these cells as microglia, a subset of astrocytes, is significant since an overabundance of microglia has been associated with the development of AD (Weizmann Institute of Science, “The brain's rejuvenating cells,” Science Daily, 9 June, 2017:sciencedaily.com/release/2017/06/170609135903).

### Example 3

### Maturation Effects of Some Amyloid Beta-Derived MRHAS Peptides on RG Cells in Culture

Peptides derived from amyloid beta and synthesized by Kinexus Inc. (Vancouver, Canada) were tested for their effects on RG cell cultures, according to the method of Yankner B R, et al., Science (1990) 250:279-282. Since septapeptides may be too small or unstable to form avid bonds with cell surface receptors, the 7 mer was bracketed within a slightly longer 10-mer construct, in order to maximize stability during the incubation procedure. A randomized sequence was used as a negative control. The sequences are summarized in Table 2.

TABLE 2
Amyloid beta-derived peptides tested for
maturation activity using RG progenitor cells
in culture: The common 7 mer is shown in
underlined, bold, capital letters and where
peptide #3 is a randomized control for peptide #2.

| Peptide #1 | Sequence |
|---|---|
| 1 | ¹DAEFRHDSGYEVHHQKLVF¹⁹ (SEQ ID NO: 16) |
| 2 | ¹³HHEQKLVFFAE²² (SEQ ID NO: 17) |
| 3 | FLVKAHEQFH (SEQ ID NO: 18) |

These peptides were dissolved in DMSO (dimethyl sulfoxide) to maintain stability. This solvent slightly altered the shape of some cells at lower dilutions of peptide, giving a high background. Therefore, to avoid any interference with the results, RG cells were incubated in the presence of peptide concentrations ranging from 10⁻⁶ M to 10⁻⁷ M for 72 hours. Positive cells were counted and data are presented as the number greater than the baseline number of maximal mature glia in untreated control cultures achieved by day 3, taken to be zero. Results are shown in Table 3 and photos of transformed cells are contained in FIG. 9.

TABLE 3
Maturation activity of amyloid beta-derived peptides on RG progenitor cells
in culture Activity shown as % conversion

| Peptide Dilution | 10⁻⁶ M | 10⁻⁷ M |
|---|---|---|
| Peptide concentration | 1 uM | 100 nM |
| Peptide #1 | 63% | 41% |
| Peptide #2 | 41% | 27% |
| Peptide #3 | <1% | <1% |

### Septapeptide Sequences

### 1. Amyloid Beta Peptide

46 amino acids,
primary constituent of senile plaques and cerebrovascular deposits in AD and Down syndrome with Septapeptide MRHAS underlined

(SEQ ID NO: 1)
¹DAEFRGDSGY EV¹³HHQKLVF¹⁹F AEDVGSNKGA IIGMVGGVVI
⁴ATVIVI

see ncbi.nlm.nih.gov/protein/AP05067.3

### 2. Major Prion Protein

256 amino acids,
cellular protein which undergoes structural isomerization to an infectious form with septapeptide MRHAS underlined

(SEQ ID NO: 2)
¹MVKSHIGSWI LVLFVAMWSD VFLCKKRPKP GGGWNTGGSR
⁴¹YPGQGSPGGN RYPPQGGGGW GQPHGGGWGQ PHGGGWGQPH
⁸¹GGGWGQPHGG GGWGQGGSHS QWNKPSKPKT NMKHMAGAAA
¹²¹AGAVVGGLGG YMLGSAMSRP LIHFGNDYED RYYRENMYRY
¹⁶¹PNQVYYRPVD QYSQNNFVHD VCVNITVKQH TVTTTTKGEN
²⁰¹FTETDMKIME RVVEQMCVTQ YQKESEAYYQ RRASAILFSS
¹⁴¹PPVILLISFL IFLIVG

see ncbi.nlm.nih.gov/protein/A018754.3
3. Human Monocyte Chemoattractant Protein (hMCP)-1
99 amino acids with secretory cytokine-like protein with chemotactic properties

(SEQ ID NO: 3)
¹MKVSAALLCL LLIAATFIPQ GLAQPDAINA PVTCCYNFTN
⁴¹RKISVQRLAS YRRITSSKCP KEAVIFKTIV AKEICADPKQ
⁸¹KWVQDSMDHL DK⁹³QTQTPKT⁹⁹

see ncbi.nlm.nih.gov/protein/AAB20651.1

TABLE 4
Summary of some MRHAS septapeptides relevant to
AD in hMCP-1, viruses, bacteria and prions

| VIRUS/BACTERIUM/ | | AMINO ACID | AMINO ACID |
|---|---|---|---|
| PRION/CHEMOKINE | PROTEIN | LOCATION | SEQUENCE |
| Rubella virus | structural | 102-108 | QPQPPRM |
| | | | (SEQ ID NO: 19) |
| Rubella virus | structural | 89-95 | QTPAPKP |
| | | | (SEQ ID NO: 20) |
| Rubella virus | structural | 313-319 | PPQPPRA |
| | | | (SEQ ID NO: 21) |
| HIV | Gag, structural | 145-151 | QAISPRT |
| | | | (SEQ ID NO: 22) |
| HIV | envelope | 655-661 | QNQQEKN |
| | | | (SEQ ID NO: 6) |
| Prion | precursor | 75-81 | QNNFVHD |
| | structural | | (SEQ ID NO: 7) |
| Amyloid beta (plaque) | structural | 13-19 | HHQKLVF |
| | | | (SEQ ID NO: 11) |
| chemokine hMCP-1 | secretory | 93-99 | QTQTPKT |
| | functional | | (SEQ ID NO: 23) |

1. A method for prophylactic or therapeutic treatment of a neurodegenerative condition in a subject which method comprises administering to said subject an agent that blocks the monocyte chemoattractant protein-1 (MCP-1) receptor on microglia precursor cells or that selectively binds to HHQKLVF (SEQ ID NO: 11).

2. The method of claim 1 wherein the agent is a tripeptide or tetrapeptide consisting of positions 1-3 or 1-4 of a septapeptide or an extended form thereof that does not include as an extension at the C-terminus the remaining amino acids of said septapeptide or is a peptidomimetic thereof or is the chaperone domain of BRIOCHS protein or is resveratrol or related flavonoid.

3. The method of claim 2 wherein the septapeptide is selected from the group consisting of the septapeptides of FIG. 1 or Table 4.

4. The method of claim 3 wherein the septapeptide is HHQKLVF (SEQ ID NO: 11).

5. The method of any of claims 1-4 wherein the neurodegenerative disease is Alzheimer's disease or Huntington's disease.

6. A method to identify an agent for prophylactic or therapeutic treatment of a neurodegenerative condition in a subject which method comprises measuring the response of microglia precursor cells or stem cells to a septapeptide an extended form thereof or binding portion thereof or extended form of said binding portion in the presence and absence of a candidate agent, wherein a candidate agent that diminishes the response of said cells to the septapeptide or portion or extended forms thereof is identified as an agent for prophylactic or therapeutic treatment of said neurodegenerative condition.

7. The method of claim 6 wherein the septapeptide is selected from the group consisting of the septapeptides of FIG. 1 or Table 4.

8. The method of claim 7 wherein the septapeptide is HHQKLVF (SEQ ID NO: 11).

9. The method of any of claims 6-8 wherein the neurodegenerative disease is Alzheimer's disease or Huntington's disease.

10. A method to identify an agent for prophylactic or therapeutic treatment of a neurodegenerative condition in a subject which method comprises contacting a candidate agent with a septapeptide or an extended form thereof and determining the presence or absence of any complex formed, whereby a candidate agent that forms said complex is identified as an agent for prophylactic or therapeutic treatment of said neurodegenerative condition.

11. The method of claim 10 wherein the septapeptide is selected from the group consisting of the septapeptides of FIG. 1 or Table 4.

12. The method of claim 11 wherein the septapeptide is HHQKLVF (SEQ ID NO: 11).

13. The method of any of claims 10-12 wherein the neurodegenerative disease is Alzheimer's disease or Huntington's disease.

14. A recombinant expression system for an extended septapeptide which comprises a nucleotide sequence encoding said extended septapeptide operably linked to heterologous control sequence to effect expression.

15. The recombinant expression system of claim 14 wherein the extended septapeptide is an extended form of septapeptides selected from the group consisting of the septapeptides of Table 4.

16. The recombinant expression system of claim 15 wherein the extended septapeptide is an extended form of septapeptide HHQKLVF (SEQ ID NO: 11).

17. A vaccine for prevention of neurodegenerative conditions caused by infectious agents which comprises as active ingredient a septapeptide or an extended form thereof along with a suitable pharmaceutically acceptable carrier.

18. The vaccine of claim 17 wherein the septapeptide is selected from the group consisting of the septapeptides of FIG. 1 or Table 4.

19. The vaccine of claim 18 wherein the septapeptide is HHQKLVF (SEQ ID NO: 11).

20. A method to determine the presence or absence of a neurodegenerative disease in a subject, which method comprises determining the presence or absence of a septapeptide or extended form thereof in the cerebral spinal fluid (CSF) of said subject, whereby the presence of said septapeptide or extended form thereof indicates the presence of neurodegenerative disease in said subject.

21. The method of claim 20 wherein said detecting comprises contacting the CSF with a binding agent for a septapeptide or extended form and detecting the presence or absence of a complex between the binding agent and a component of said CSF.

22. The method of claim 21 wherein the binding agent is an antibody or an aptamer.

23. The method of claim 21 wherein the septapeptide is HHQKLVF (SEQ ID NO: 11).

24. The method of any of claims 20-33 wherein the neurodegenerative disease is Alzheimer's disease or Huntington's disease.

25. A binding moiety that is specifically reactive with a septapeptide or extended form thereof wherein the septapeptide is selected from the group consisting of those shown in Table 4.

26. The binding moiety of claim 25 which is an antibody or antigen binding portion thereof or which is an aptamer.

27. The binding moiety of claim 25 or 26 wherein the septapeptide is HHQKLVF (SEQ ID NO: 11).


SEQUENCE LISTING (23 sequences)

SEQ ID NO: 1 - protein, 46 aa, Homo sapiens
DAEFRGDSGYEVHHQKLVFFAEDVGSNKGAIIGMVGGVVIATVIVI

SEQ ID NO: 2 - protein, 256 aa, Homo sapiens
MVKSHIGSWILVLFVAMWSDVFLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHMAGAAAAGAVVGGLGGYMLGSAMSRPLIHFGNDYEDRYYRENMYRYPNQVYYRPVDQYSQNNFVHDVCVNITVKQHTVTTTTKGENFTETDMKIMERVVEQMCVTQYQKESEAYYQRRASAILFSSPPVILLISFLIFLIVG

SEQ ID NO: 3 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 4 - protein, 11 aa, Artificial Sequence
  Synthetic
GSNKGAIIGLN

SEQ ID NO: 5 - protein, 4 aa, Artificial Sequence
  Synthetic
HHQK

SEQ ID NO: 6 - protein, 7 aa, Human immunodeficiency virus
QNQQEKN

SEQ ID NO: 7 - protein, 7 aa, Homo sapiens
QNNFVHD

SEQ ID NO: 8 - protein, 7 aa, Escherichia coli
NNGPTHE

SEQ ID NO: 9 - protein, 8 aa, Artificial Sequence
  Synthetic
NNAAATHE

SEQ ID NO: 10 - protein, 7 aa, Artificial Sequence
  Synthetic
NNGPAAA

SEQ ID NO: 11 - protein, 7 aa, Homo sapiens
HHQKLVF

SEQ ID NO: 12 - protein, 7 aa, Listeria monocytogenes
QQTAPKA

SEQ ID NO: 13 - protein, 7 aa, Haemophilus influenzae
QVQNNKP

SEQ ID NO: 14 - protein, 7 aa, Streptococcus pneumoniae
QQQPPKA

SEQ ID NO: 15 - protein, 10 aa, Homo sapiens
HHQKLVFFAE

SEQ ID NO: 16 - protein, 19 aa, Artificial Sequence
  Synthetic
DAEFRHDSGYEVHHQKLVF

SEQ ID NO: 17 - protein, 10 aa, Artificial Sequence
  Synthetic
HHQKLVFFAE

SEQ ID NO: 18 - protein, 10 aa, Artificial Sequence
  Synthetic
FLVKAHEQFH

SEQ ID NO: 19 - protein, 7 aa, Rubella virus
QPQPPRM

SEQ ID NO: 20 - protein, 7 aa, Rubella virus
QTPAPKP

SEQ ID NO: 21 - protein, 7 aa, Rubella virus
PPQPPRA

SEQ ID NO: 22 - protein, 7 aa, Human immunodeficiency virus
QAISPRT

SEQ ID NO: 23 - protein, 7 aa, Homo sapiens
QTQTPKT